SEQUENCE LISTING (20 sequences)

SEQ ID NO: 1 - protein, 408 aa, T. forsythia
MKTQWMCIGIMALMGACTSDQETPKPLTEAHPIILKKAEKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVKTSSPSTTPINFHINKPFVFAIREKSTGVILFIGEIGEVKE

SEQ ID NO: 2 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVKTSSPSTTPINFHINKPFVFAIREKSTGVILFIGEIGEVKE

SEQ ID NO: 3 - protein, 10 aa, Artificial Sequence
  Synthetic Construct
TAVEMRVKSS

SEQ ID NO: 4 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMRVKSSPSTTPINFHINKPFVFAIREKSTGVILFIGEIGEVKE

SEQ ID NO: 5 - protein, 10 aa, Artificial Sequence
  Synthetic Construct
TAVEMVRTSS

SEQ ID NO: 6 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVRTSSPSTTPINFHINKPFVFAIREKSTGVILFIGEIGEVKE

SEQ ID NO: 7 - protein, 10 aa, Artificial Sequence
  Synthetic Construct
TAVEMVARSS

SEQ ID NO: 8 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVARSSPSTTPINFHINKPFVFAIREKSTGVILFIGEIGEVKE

SEQ ID NO: 9 - protein, 10 aa, Artificial Sequence
  Synthetic Construct
TAVEMVFTSS

SEQ ID NO: 10 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVFTSSPSTTPINFHINKPFVFAIREKSTGVILFIGEIGEVKE

SEQ ID NO: 11 - protein, 10 aa, Artificial Sequence
  Synthetic Construct
TAVEMVETSS

SEQ ID NO: 12 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVETSSPSTTPINFHINKPFVFAIREKSTGVILFIGEIGEVKE

SEQ ID NO: 13 - protein, 10 aa, Artificial Sequence
  Synthetic Construct
TAVEMVDTSS

SEQ ID NO: 14 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVDTSSPSTTPINFHINKPFVFAIREKSTGVILFIGEIGEVKE

SEQ ID NO: 15 - protein, 10 aa, Artificial Sequence
  Synthetic Construct
TAVEMVNTSS

SEQ ID NO: 16 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVNTSSPSTTPINFHINKPFVFAIREKSTGVILFIGEIGEVKE

SEQ ID NO: 17 - protein, 370 aa, Artificial Sequence
  Synthetic Construct
  SITE (324)..(333): Xaa 324 to 333 are any amino acids other than Thr-Ala-Val-Glu-Met-Val-Lys-Thr-Ser-Ser
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVXXXXXXXXXXPSTTPINFHINKPFVFAIREKSTGVILFIGEIGEVKE

SEQ ID NO: 18 - protein, 10 aa, Artificial Sequence
  Synthetic Construct
TAVEMVKTSS

SEQ ID NO: 19 - protein, 10 aa, Artificial Sequence
  Synthetic Construct
TAVEMVATSS

SEQ ID NO: 20 - protein, 370 aa, Artificial Sequence
  Synthetic Construct
EKIEKDNAFAFDLLQTTRKHVTEANVFISPLSVSMALNMTLNGAAGVTADEMKTALRETGYTMEDINEYSHSLREALLKVDPSTTIGMANSIWYKQGELVKEPFILANRTHYDAEVKAVDFSSPATLPAINGWCARKTNDKITKILDYIPGNAFMYLINAVYFKGIWVTQFKKSDTKRAPFRKADGTTQEVNMMAQKSTFGYTTDECCQYLEMDYGNKAFSMIVMLPNEGQTTRDVIEQLDNKHWSMIIKGIRPTQVSLRMPRFKTECKYGLEKKILPEMGMNVPFTETADFPGITDAAIFISRVIHKTFVQVDEEGTEAAAVTAVEMVATSSPSTTPINFHINKPFVFAIREKSTGVILFIGEIGEVKE

(19) United States
(12) Patent Application Publication
(10) Pub. No.: US 2024/0051991 A1
(43) Pub. Date: Feb. 15, 2024

(54) Recombinant Miropin

(71) Applicants: UNIVERSITY OF LOUISVILLE, Louisville, KY (US); JAGIELLONIAN UNIVERSITY, Krakow (PL)

(72) Inventors: Jan S. Potempa, Louisville, KY (US); Miroslaw Ksiazek, Wadowice (PL); Danuta M. Mizgalska, Krakow (PL); Malgorzata L. Benedyk-Machaczka, Krakow (PL)

(21) Appl. No.: 18/555,877

(22) PCT Filed: Mar. 16, 2022
(86) PCT No.: PCT/US2022/071178

§ 371 Date: Oct. 27, 2023

Related U.S. Application Data
(60) Provisional application No. 63/176,945, filed on Apr. 20, 2021.

(51) Int. Cl. C07K 4/04; A61P 31/04

(52) U.S. Cl. CPC C07K 4/04; A61P 31/04; A61K 38/00

(57) ABSTRACT

Disclosed herein are recombinant miropin polypeptides capable of inhibiting a broad spectrum of proteases, including Kgp and Rgp gingipains, trypsin, plasmin, elastase, cathepsin G, cathepsin L, and plasma kallikrein. Also disclosed herein are methods of treating a disease or condition in a subject that involves administering to the subject a recombinant miropin disclosed herein.

### CROSS-REFERENCE TO RELATED APPLICATIONS

This application claims benefit of U.S. Provisional Application No. 63/176,945, filed Apr. 20, 2021, which is hereby incorporated herein by reference in its entirety.

### SEQUENCE LISTING

This application contains a sequence listing filed in electronic form as an ASCII.txt file entitled “321207_2010_Sequence_Listing_ST25” created on Mar. 15, 2022, having 38,417 bytes. The content of the sequence listing is incorporated herein in its entirety.

### BACKGROUND

Periodontal diseases are bacterial-associated inflammatory diseases of the supporting tissues of the teeth and range from the relatively mild form of gingivitis, the non-specific, reversible inflammation of gingival tissue to the more severe forms of periodontitis which are characterized by the destruction of the tooth's supporting alveolar bone structures. Periodontitis is caused by a subgingival infection of a consortium of specific Gram-negative bacteria that leads to the destruction of the periodontium and is a major public health problem. One bacterium that has attracted considerable interest is P. gingivalis as the recovery of this microorganism from adult periodontitis lesions can be up to 50% of the subgingival anaerobically cultivable flora, whereas P. gingivalis is rarely recovered, and then in low numbers, from healthy sites. A proportional increase in the level of P. gingivalis in subgingival plaque has been associated with an increased severity of periodontitis and eradication of the microorganism from the cultivable subgingival microbial population is accompanied by resolution of the disease. The progression of periodontitis lesions in non-human primates has been demonstrated with the subgingival implantation of P. gingivalis. These findings in both animals and humans suggest P. gingivalis plays a major role in the development of adult periodontitis. Thus, it is not a surprise that P. gingivalis is considered/named as a “keystone” pathogen in the pathogenicity of periodontitis.

Arg-gingipains (RgpA and RgpB) and Lys-gingipain (Kgp) are endopeptidase enzymes secreted by P. gingivalis. These gingipains serve many functions for the organism, contributing to its survival and virulence. Arg-gingipains have been found to play a key role in the collection of nutrients necessary for P. gingivalis survival. Rgp degrades a large number of host proteins, including human serum albumin and fibrinogen, this providing this asaccharolytic bacterium with an abundant nitrogen and carbon source from generated peptides. The gingipains are also responsible for a number of necessary functions related to host invasion, colonization and evasion of host defense systems. Rgps are also responsible for processing of precursor proteins of fimbriae, which are involved in adhesion and invasion of host cells.

Gingipains are key factors in tissue damage symptoms of periodontitis, which results among others from direct or indirect the degradation of proteinaceous components of matrix, such as collagen, and fibronectin. Degradation of these substrates interferes with interactions between host cells and the extracellular matrix, therefore impeding wound healing and causing destruction of periodontal tissues. Rgps are responsible for eliciting the host inflammatory response via the p38α MAPK transduction pathway. This response likely contributes to the inflammatory nature of periodontitis and is involved in tissue and bone destruction.

Gingipains have been associated with Alzheimer's disease (AD). Gingipains were identified in tissue microarrays (TMAs) containing brain tissue cores from the middle temporal gyrus (MTG) of patients exhibiting AD brain pathology. Both RgpB and Kgp were discovered in the hippocampus and cerebral cortex of AD patients and were found to be associated with tau load, a marker for AD pathology and ubiquitin, which accumulates in tau tangles and amyloid beta plaques in AD brain. P. gingivalis 16S rRNA was also discovered in the cerebral cortex and cerebrospinal fluid (csf) of AD brains. Pretreatment with gingipains inhibitors protected neuron cell degradation caused by oral administration of gingipains secreting W83 strain of P. gingivalis in murine model.

### SUMMARY

The gram negative bacteria Tannerella forsythia secretes a protease inhibitor belonging to serpin (serine protease inhibitor) superfamily referred to herein as “miropin”. In striking contrast to other serpins, miropin efficiently inhibits a broad range of serine and cysteine proteases.

Disclosed herein are recombinant miropin polypeptides capable of inhibiting a broad spectrum of proteases, including Kgp and Rgp gingipains, trypsin, plasmin, elastase, cathepsin G, cathepsin L, and plasma kallikrein. Also disclosed herein are methods of treating a disease or condition in a subject that involves administering to the subject a recombinant miropin disclosed herein.

There are several possible ways to apply miropin as the therapeutic. For example, the polypeptide can be added to a mouth wash, toothpaste, or chewing gum.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1F show recombinant miropin is a potent irreversible Kgp inhibitor (FIGS. 1A, 1B and 1C), whereas VRT-miropin (FIGS. 1D, 1E, and 1F), and RVK-miropin variants inhibit in the same manner, respectively, RgpB (FIGS. 1C, 1D, and 1E) and both Kgp and Rgp gingipains in P. gingivalis (Pg) cultures (FIG. 1G). FIGS. 1A and 1D show stoichiometry of Kgp (FIG. 1A) and RgpB (FIG. 1D) inhibition, respectively. FIGS. 1B and 1E show progression curve analysis of Kgp (FIG. 1B) and RgpB (FIG. 1E) inhibition, respectively. Gingipains were added to mixtures containing a constant amount of substrate (S) and increasing concentrations of miropin. Changes in absorbance (Abs) at 410 nm was then recorded. The number associated with each progress curve represents the concentration of miropin (nM). FIGS. 1C and 1F show k_{obs} values for Kgp (FIG. 1C) and RgpB (FIG. 1F) plotted as a function of miropin concentration, and kₐₛₛ was determined from the slope of a linear curve fitted to the data points and corrected for the stoichiometry and [S]/K_{M} factor. FIG. 1G shows residual activity of Rgps and Kgp in Pg culture preincubated with RVK-miropin at 100 nM final concentration. Activity in cultures without added inhibitor was arbitrary taken as 100%

FIG. 2A shows stoichiometry of inhibition. FIG. 2B shows progression curve analysis of Kgp inhibition by miropin. Kgp was added to mixtures containing a constant amount of substrate and increasing concentrations of miropin. Changes in absorbance (Abs) then recorded. The number associated with each progress curve represents the concentration of miropin (nM). FIG. 2C shows k_{obs} plotted as a function of miropin concentration, and kₐₛₛ determined from the slope of a linear curve fitted to the data points and corrected for the stoichiometry factor and [S]/K_{M} factor.

FIGS. 3A to 3D show Tannerella forsythia (Tf) in co-infection with Pg prevents mouse mortality in a miropin-dependent manner, apparently by inhibiting Kgp activity. Subcutaneous chambers were inoculated with wt Pg (strain W83), wt-Tf, miropin-null Tf (Δmir), or Kgp-deficient Pg W83 (Δkgp) alone at 10⁹ CFU or in combinations: wt−Pg+wt Tf and wt−Pg+Δmir. Mortality of infected mice was recorded for 6 days (FIG. 3A). Twenty-four hours after infection, 20 μL of chamber content was withdrawn and analyzed for live Pg content by plating and colony formation assay (FIG. 3B). Rgp-specific (FIG. 3C) and Kgp-specific (FIG. 3D) activities were determined using Bz-Arg-pNA (BApNA) and Tos-Gly-Pro-Lys-pNA as a substrate, respectively. Significance of differences was calculated by one-way ANOVA. *p<0.05, **p<0.01, ***p<0.001.

FIGS. 4A to 4F show miropin is a lipoprotein located on the cell surface of Tf. FIG. 4A shows the N-terminal amino-acid sequence (SEQ ID NO:1) of miropin, with a canonical signal peptide (lower case), lipobox (lowercase and bold font), extension (underlined) that presumably crosses the S-layer, and the beginning of the serpin domain. FIG. 4B shows TEM of a Tf cell with cell membrane (CM), outer membrane (OM), and S-layer (S) indicated by arrows. FIG. 4C shows dot-blot analysis of intact and lysed wild-type Tf and a Δmir strain using rabbit polyclonal antibody (pAb) against miropin and streptavidin conjugated to horseradish peroxidase. Biotinylated protein(s) are biomarkers for the IM. FIG. 4D shows flow cytometry analysis with anti-miropin pAb. FIG. 4E shows titration of Pg cell surface-associated Kgp activity with recombinant miropin. FIG. 4F shows inhibition of Kgp on the Pg surface by a suspension of intact Tf cells. Activity of Pg cell suspension alone was arbitrary taken as 100%.

FIG. 5 show that growth of P. gingivalis W83 (Pg wt) in minimal medium (2% BSA in DMED) was completely inhibited by 5 μM recombinant miropin RVK. Noteworthy, miropin VAT, inhibiting neither Rgps nor Kgp, had no effect on Pg growth. As expected, Pg ΔKRAB, a strain not secreting any gingipains, did not grow in minimal medium.

FIGS. 6A to 6D show recombinant miropin prevents mice mortality induced by Pg infection in an inhibitor specificity-dependent manner. Subcutaneous chambers injected with 250 μg wt-miropin (VKT), or VFT-miropin or RVK-miropin or PBS were inoculated with Pg (strain W83, 1×10⁹ CFU). FIG. 6A shows mortality of infection recorded for 6 days. FIGS. 6B to 6D show analysis of 20 μL of chamber content was withdrawn twenty-four hours after infection and analyzed for: live Pg content (CFU) by plating and colony formation assay (FIG. 6B); Kgp activity (FIG. 6C); and Rgp activity (FIG. 6D).

FIG. 7 shows lung co-infection with T. forsythia reduced mice mortality caused by P. gingivalis. Lungs were inoculated with indicated CFU of bacteria and mice survival was recorded.

FIG. 8 shows lung co-infection with T. forsythia prevented P. gingivalis-induced lung damage illustrated by histological examination of the lung tissue.

FIG. 9 shows miropin quenches P. gingivalis (Pg)-induced proinflammatory response in infected lungs and contributes to the lack of non-pathogenic character of T. forsythia (Tf). VVT=wild type Tf; Tf miropin=Δmiropin Tf.

FIG. 10 shows miropin expression attenuates T. forsythia ability to cause lung damage as illustrated by histological examination of the lung tissue of animals infected with WT-Tf and isogenic mutant deficient of miropin (Δmir).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Disclosed herein are recombinant miropin polypeptides capable of inhibiting a broad spectrum of proteases, including Kgp and Rgp gingipains. As disclosed herein, a recombinant miropin comprising the amino acid sequence TAVEMX₁X₂X₃SS (SEQ ID NO:11), wherein if X₁ is Val, X₂ is not Lys and/or X₃ is not Thr, and wherein if X₂ is Lys, X₁ is not Val and/or X₃ is not Thr, and wherein if X₃ is Thr, X₁ is not Val and/or X₂ is not Lys, inhibits different proteases than wildtype miropin.

Full length wildtype miropin for the T. forsythia strain ATCC43037 (Gen-Bank™ code WP_041590947; UniProt code G8UQY8) is set forth in SEQ ID NO:1. Lower case designates the signal peptide.

(SEQ ID NO: 1)
mktqwmcigimalmgaCTSDQETPKPLTEAHPIILKKAEKIEKDNAFAFD
LLQTTRKHVTEANVFISPLSVSMALNMTLNGAAGVTADEMKTALRETGYT
MEDINEYSHSLREALLKVDPSTTIGMANSIWYKQGELVKEPFILANRTHY
DAEVKAVDFSSPATLPAINGWCARKTNDKITKILDYIPGNAFMYLINAVY
FKGIWVTQFKKSDTKRAPFRKADGTTQEVNMMAQKSTFGYTTDECCQYLE
MDYGNKAFSMIVMLPNEGQTTRDVIEQLDNKHWSMIIKGIRPTQVSLRMP
RFKTECKYGLEKKILPEMGMNVPFTETADFPGITDAAIFISRVIHKTFVQ
VDEEGTEAAAVTAVEMVKTSSPSTTPINFHINKPFVFAIREKSTGVILFI
GEIGEVKE.

Recombinant miropin spanning residues Glu39—Glu408 was produced from a construct derived from plasmid pGEX-6P-1_2.2 (Goulas et al. (2017) J Biol Chem 292, 10883-10898), which attaches an N-terminal glutathione S-transferase (GST) tag and a PreScission protease cleavage site. Note that the R174Q mutation is attributed to natural variability in forsythia strains.

(SEQ ID NO: 2)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMVKTSSPSTTPINFHINKPFVFA
IREKSTGVILFIGEIGEVKE.

Comparing to miropin in the genome of T. forsythia, the recombinant miropin is already shortened by 16 amino acids signal peptide for lipoproteins and 22 amino acids extension serving as a spacer enabling miropin to be located above of S-layer. Therefore, in some embodiments, the disclosed recombinant miropin contains only the core sequence described for inhibitory serpins.

Miropin shares the structure (or fold) withal other serpins which includes three β-sheets (referred to as A, B, and C) and 8 α-helices (hA-hH). For serpin function the most significant are A-sheet and the RCL. Most of residues in these structures are critical and this especially applies to strands A3 and A5 (s3A and s5A) of b-sheet A. Changes in other structural elements, except residues essential for the proper fold of serpin, can be tolerated. The unique feature of the miropin fold is plasticity of sheet A allowing accommodation of extra b-strand of variable length.

Miropin specificity is dictated by the amino acid sequence TAVEMVKTSS (SEQ ID NO:18) constituting the exposed loop of the RCL. Amino acid residues substitutions in this segment can lead to changes in inhibitory spectrum miropin.

Therefore, in some embodiments, the recombinant miropin comprises the amino acid sequence SEQ ID NO:1 or 2, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1 or 2, wherein the recombinant miropin comprises the amino acid sequence TAVEMX₁X₂X₃SS (SEQ ID NO:11), wherein if X₁ is Val, X₂ is not Lys and/or X₃ is not Thr, and wherein if X₂ is Lys, X₁ is not Val and/or X₃ is not Thr, and wherein if X₃ is Thr, X₁ is not Val and/or X₂ is not Lys.

In some embodiments, the recombinant miropin comprises the amino acid sequence TAVEMRVKSS (SEQ ID NO:3), which inhibits trypsin, plasmin, neutrophil elastase, plasma kallikrein, Kgp, and Rgp. Therefore, in some embodiments, the recombinant miropin comprises the amino acid sequence:

(SEQ ID NO: 4)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMRVKSSPSTTPINFHINKPFVFA
IREKSTGVILFIGEIGEVKE,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:4 that comprises the amino acid sequence TAVEMRVKSS (SEQ ID NO:3).

In some embodiments, the recombinant miropin comprises the amino acid sequence TAVEMVRTSS (SEQ ID NO:5), which inhibits trypsin, plasmin, neutrophil elastase, cathepsin G, cathepsin L, and Rgp. Therefore, in some embodiments, the recombinant miropin comprises the amino acid sequence:

(SEQ ID NO: 6)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMVRTSSPSTTPINFHINKPFVFA
IREKSTGVILFIGEIGEVKE,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:6 that comprises the amino acid sequence comprises the amino acid sequence TAVEMVRTSS (SEQ ID NO:5).

In some embodiments, the recombinant miropin comprises the amino acid sequence TAVEMVARSS (SEQ ID NO:7), which inhibits trypsin, plasmin, cathepsin L. Therefore, in some embodiments, the recombinant miropin comprises the amino acid sequence:

(SEQ ID NO: 8)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMVARSSPSTTPINFHINKPFVFA
IREKSTGVILFIGEIGEVKE,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:8 that comprises the amino acid sequence comprises the amino acid sequence TAVEMVARSS (SEQ ID NO:7).

In some embodiments, the recombinant miropin comprises the amino acid sequence TAVEMVFTSS (SEQ ID NO:9), which inhibits neutrophil elastase, cathepsin G, cathepsin L, and cathepsin K. Therefore, in some embodiments, the recombinant miropin comprises the amino acid sequence:

(SEQ ID NO: 10)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMVFTSSPSTTPINFHINKPFVFA
IREKSTGVILFIGEIGEVKE,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:10 that comprises the amino acid sequence comprises the amino acid sequence TAVEMVFTSS (SEQ ID NO:9).

In some embodiments, the recombinant miropin comprises the amino acid sequence TAVEMVETSS (SEQ ID NO:11). Therefore, in some embodiments, the recombinant miropin comprises the amino acid sequence:

(SEQ ID NO: 12)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMVETSSPSTTPINFHINKPFVFA
IREKSTGVILFIGEIGEVKE,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:12 that comprises the amino acid sequence comprises the amino acid sequence TAVEMVARSS (SEQ ID NO:11).

In some embodiments, the recombinant miropin comprises the amino acid sequence TAVEMVDTSS (SEQ ID NO:13). Therefore, in some embodiments, the recombinant miropin comprises the amino acid sequence:

(SEQ ID NO: 14)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMVDTSSPSTTPINFHINKPFVFA
IREKSTGVILFIGEIGEVKE,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:14 that comprises the amino acid sequence comprises the amino acid sequence TAVEMVARSS (SEQ ID NO:13).

In some embodiments, the recombinant miropin comprises the amino acid sequence TAVEMVNTSS (SEQ ID NO:15). Therefore, in some embodiments, the recombinant miropin comprises the amino acid sequence:

(SEQ ID NO: 16)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMVNTSSPSTTPINFHINKPFVFA
IREKSTGVILFIGEIGEVKE,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:16 that comprises the amino acid sequence comprises the amino acid sequence TAVEMVARSS (SEQ ID NO:15).

In some embodiments, the recombinant miropin comprises the amino acid sequence TAVEMVATSS (SEQ ID NO:19), which inhibits elastase. Therefore, in some embodiments, the recombinant miropin comprises the amino acid sequence:

(SEQ ID NO: 20)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVTAVEMVATSSPSTTPINFHINKPFVFA
IREKSTGVILFIGEIGEVKE,

or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:16 that comprises the amino acid sequence comprises the amino acid sequence TAVEMVARSS (SEQ ID NO:19).

In some embodiments, the recombinant miropin comprises the amino acid sequence:

(SEQ ID NO: 17)
EKIEKDNAFAFDLLQTTRKHVTEANVFISPLSVSMALNMTLNGAAGVTAD
EMKTALRETGYTMEDINEYSHSLREALLKVDPSTTIGMANSIWYKQGELV
KEPFILANRTHYDAEVKAVDFSSPATLPAINGWCARKTNDKITKILDYIP
GNAFMYLINAVYFKGIWVTQFKKSDTKRAPFRKADGTTQEVNMMAQKSTF
GYTTDECCQYLEMDYGNKAFSMIVMLPNEGQTTRDVIEQLDNKHWSMIIK
GIRPTQVSLRMPRFKTECKYGLEKKILPEMGMNVPFTETADFPGITDAAI
FISRVIHKTFVQVDEEGTEAAAVX₁X₂X₃X₄X₅X₆X₇X₈X₉X₁₀PSTTPINFHI
NKPFVFAIREKSTGVILFIGEIGEVKE,

where X₁-X₁₀ are any amino acid combination other than TAVEMVKTSS (SEQ ID NO:18). For example, X₁-X₁₀ can be the amino acid sequence TAVEMVKTSS (SEQ ID NO:18) but with at least 1, 2, 3, 4, 5, 6, 7, 8, or 9 amino acid substitutions. In some embodiments, the recombinant miropin is a variant having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:17.

Also disclosed herein are methods of treating a disease or condition in a subject that involves administering to the subject a recombinant miropin disclosed herein. Table 1 provides a list of pathological conditions with known target proteases.

TABLE 1

| Targeted protease | Pathological condition |
|---|---|
| N. Elastase | Chronic inflammatory lung diseases |
| | Chronic obstructive inflammatory diseases |
| | Acute lung injury |
| | Acute respiratory distress syndrome |
| | Cystic fibrosis |
| Cathepsin G | P. aeruginosae lung infection |
| | Arthritis |
| Proteinase 3 (Pr3) | Atherosclerosis |
| | Lupus |
| | Asthma |
| | Treatment post-transplantation (liver, . . .) |
| NSP4 | Tumor/cancer |
| | Psoriasis |
| Thrombin | Blood coagulation |
| | Oncology/cancer |
| | Hepatic inflammation/fatty liver disease |
| Plasmin | Thrombose |
| | Cancer |
| | Proliferative Vitreoretinopathy |
| | a2-plasmin-inhibitor deficiency (Miyasato |
| | disease) |
| Cathepsin K | Osteoporosis |
| | Atherosclerosis |
| | Arthritics |
| | Diabetes and obesity |
| | Psoriasis |
| | Tumor/cancer |
| | Osteoarthritis |
| | aortic stenosis |
| | aneurism |
| | Lupus? |
| Cathepsin S | Rheumatoid Arthritics |
| | Atherosclerosis |
| | Asthma |
| | Cystic fibrosis |
| | COPD |
| | Lupus and auto-immune diseases |
| | Allergic inflammation |
| | Diabetic nephropathy and obesity |
| | Psoriasis |
| | neuropathic pain/pain |
| | Multiple sclerosis |
| | Primary Sjogren syndrome |
| | abdominal aortic aneurysm |
| | Tumor/cancer |
| | aortic stenosis |
| | aneurism |
| Cathepsin V/L | Viral infection (SARS-coV, EBOV, HeV, |
| | NiV, etc) |
| | Tumor/cancer |
| | Arthritis |
| | hair loss |
| | autoimmune disease |
| | aortic stenosis |
| | aneurism |
| | Atherosclerosis |
| Cathepsin B | Rheumatoid Arthritics |
| | Tumor/cancer |
| | Inflammatory airway disease |
| | COPD |
| | osteoarthritis |
| | Pneumocystis carinii |
| | acute pancreatitis |
| | Bone and joint disorders |
| | Asthma |
| | Atherosclerosis |
| | multiple sclerosis |
| | Psoriasis |
| | Liver fibrosis |
| | Pancreatitis |
| | Cerebral ischemia |
| | Osteoarthritis |
| | immune disease |
| | Neurodegenerative disease |
| | Alzheimer's |
| Plasma kallikreins | hereditary angioedema |
| | edema |
| | diabetic retinopathy |
| Tissue kallikreins | skin |
| | Airway, renal, cardiovascular |
| Insect serine protease | Biocontrol in Insect pest control |
| Human digestive | Crohn disease |
| proteases: | Irritate bowel syndrome |
| Trypsin, chymotrypsin and | |
| Pancreatic elastase | |
| Dentilisin of Treponema | Human periodontitis |
| denticola | |
| V8 protease of | Minor skin infections such as pimple, |
| Staphylococcus aureus | abscesses, impetigo, pneumonia, wound |
| | infection, hospital-acquired infections |
| Lys/Arg-specific channel | Cystic fibrosis |
| activating proteases | |
| (CAPs): | |
| prostasin, matriptase, and | |
| furin | |

Also disclosed is pharmaceutical compositions containing therapeutically effective amounts of one or more of the disclosed recombinant miropin polypeptide and a pharmaceutically acceptable carrier. Pharmaceutical carriers suitable for administration of the polypeptides provided herein include any such carriers known to those skilled in the art to be suitable for the particular mode of administration.

In addition, the polypeptides may be formulated as the sole pharmaceutically active ingredient in the composition or may be combined with other active ingredients. For example, the polypeptides may be formulated or combined with known NSAIDs, anti-inflammatory compounds, steroids, and/or antibiotics.

The compositions contain one or more recombinant miropin polypeptides provided herein. The polypeptides are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the polypeptides are formulated into pharmaceutical compositions using techniques and procedures well known in the art.

In one embodiment, the compositions are formulated for single dosage administration. To formulate a composition, the weight fraction of polypeptides is dissolved, suspended, dispersed or otherwise mixed in a selected carrier at an effective concentration such that the treated condition is relieved, or one or more symptoms are ameliorated.

The active polypeptides are included in the pharmaceutically acceptable carrier in an amount sufficient to exert a therapeutically useful effect in the absence of undesirable side effects on the patient treated. The therapeutically effective concentration may be determined empirically by testing the polypeptides in in vitro, ex vivo and in vivo systems, and then extrapolated therefrom for dosages for humans.

The concentration of active polypeptides in the pharmaceutical composition will depend on absorption, inactivation and excretion rates of the active polypeptides, the physicochemical characteristics of the polypeptides, the dosage schedule, and amount administered as well as other factors known to those of skill in the art.

Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

In instances in which the polypeptides exhibit insufficient solubility, methods for solubilizing polypeptides may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Liquid pharmaceutically administrable compositions can, for example, be prepared by dissolving, dispersing, or otherwise mixing an active polypeptides as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%- 100% active ingredient, or in one embodiment 0.1-95%.

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene !aural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The polypeptides could be provided in a composition that protects it from the acidic environment of the stomach. For example, the composition can be formulated in an enteric coating that maintains its integrity in the stomach and releases the active polypeptides in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The polypeptides can also be administered as a component of an elixir, suspension, syrup, wafer, sprinkle, chewing gum or the like. A syrup may contain, in addition to the active polypeptides, sucrose as a sweetening agent and certain preservatives, dyes and colorings and flavors.

The active materials can also be mixed with other active materials which do not impair the desired action, or with materials that supplement the desired action. The active ingredient is a polypeptide as described herein. Higher concentrations, up to about 98% by weight of the active ingredient, may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water- in-oil. For example, the polypeptide can be added to a mouth wash, toothpaste, or chewing gum.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. Fora liquid dosage form, the solution, e.g., for example, in a polyethylene glycol, may be diluted with a sufficient quantity of a pharmaceutically acceptable liquid carrier, e.g. , water, to be easily measured for administration. Alternatively, liquid or semi-solid oral formulations may be prepared by dissolving or dispersing the active polypeptides in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a polypeptide provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates. Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Parenteral administration, in one embodiment characterized by injection, either subcutaneously, intramuscularly or intravenously is also contemplated herein.

Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (See, e.g., U.S. Pat. No. 3,710,795) is also contemplated herein. Briefly, a polypeptide provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxy ethanol copolymer, that is insoluble in body fluids. The polypeptides diffuse through the outer polymeric membrane in a release rate controlling step. The percentage of active polypeptides contained in such parenteral compositions is highly dependent on the specific nature thereof, as well as the activity of the polypeptides and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The concentration of the pharmaceutically active polypeptides is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration should be sterile, as is known and practiced in the art.

Illustratively, intravenous or intraarterial infusion of a sterile aqueous solution containing an active polypeptide is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active polypeptide to the treated tissue(s).

The polypeptides may be suspended in micronized or other suitable form or may be derivatized to produce a more soluble active product or to produce a prodrug. The form of the resulting mixture depends upon a number of factors, including the intended mode of administration and the solubility of the polypeptides in the selected carrier or vehicle. The effective concentration is sufficient for ameliorating the symptoms of the condition and may be empirically determined.

Of interest herein are also lyophilized powders, which can be reconstituted for administration as solutions, emulsions and other mixtures. They may also be reconstituted and formulated as solids or gels.

The sterile, lyophilized powder is prepared by dissolving a polypeptides provided herein in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the polypeptides. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

In some embodiments, the disclosed recombinant miropin polypeptides are expressed, secreted, surface displayed and/or released by bacteria. The bacterial delivery vector may be attenuated, non-pathogenic, low pathogenic (including wild type), or a probiotic bacterium. The bacteria are introduced either systemically (e.g., parenteral, intravenous (IV), intramuscular (IM), intralymphatic (IL), intradermal (ID), subcutaneously (sub-q), local-regionally (e.g., intralesionally, intratumorally (IT), intraperitoneally (IP), topically, intathecally (intrathecal), by inhaler or nasal spray) or to the mucosal system through oral, nasal, pulmonary intravessically, enema or suppository administration where they are able to undergo limited replication, express, surface display, secrete and/or release the recombinant miropin polypeptides, and thereby provide a therapeutic benefit.

Bacterial vectors include non-pathogenic bacteria of the gut such as E. coli strains, Bacteroides, Bifidobacterium and Bacillus, attenuated pathogenic strains of E. coli including enteropathogenic and uropathogenic isolates, Enterococcus sp. and Serratia sp. as well as attenuated Shigella sp., Yersinia sp., Streptococcus sp. and Listeria sp. Bacteria of low pathogenic potential to humans such as Clostridium spp. and attenuated Clostridium spp., Proteus mirabilis, insect pathogenic Xenorhabdus sp., Photorhabdus sp. and human wound Photorhabdus (Xenorhabdus) are also encompassed. Probiotic strains of bacteria are also encompassed, including Lactobacillus sp., Lactococcus sp., Leuconostoc sp., Pediococcus sp., Streptococcus sp., Streptococcus agalactiae, Lactococcus sp., Bacillus sp., Bacillus natto, Bifidobacterium sp., Bacteroides sp., and the 1917 Nissel strain.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

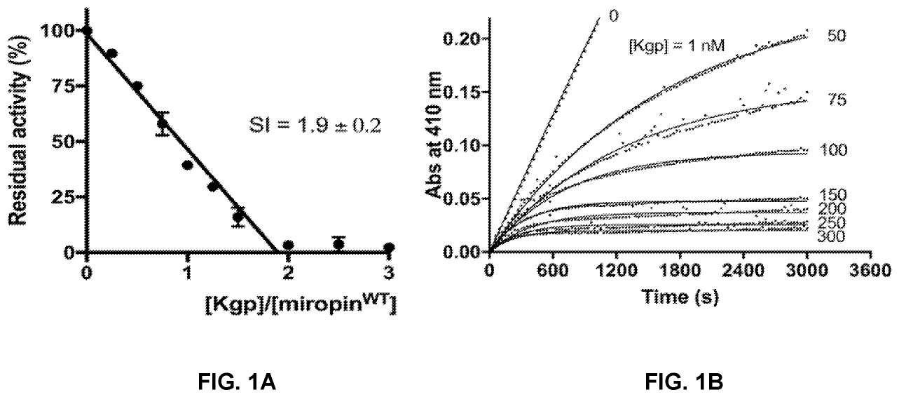

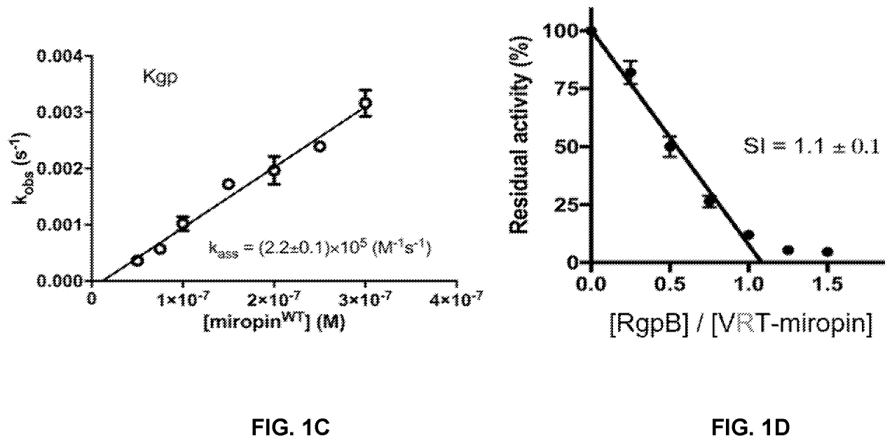

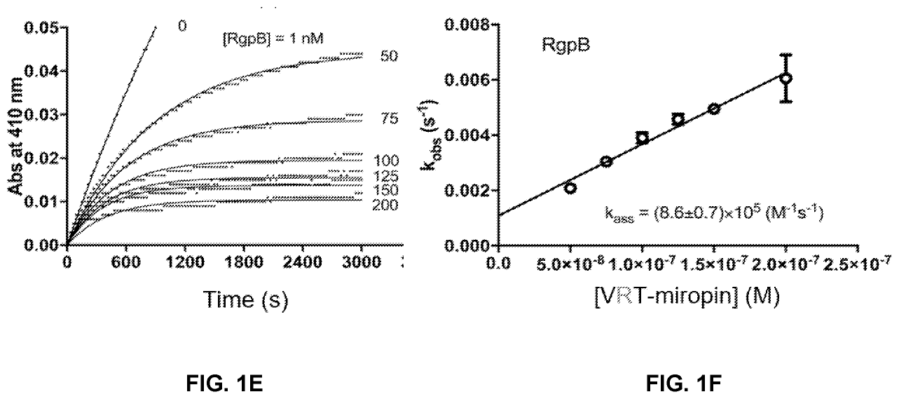

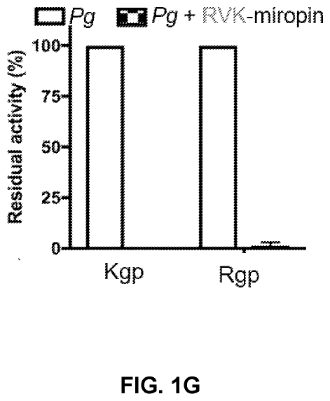

Tannerella forsythia secretes a protease inhibitor belonging to serpin (serine protease inhibitor) superfamily referred to herein as miropin. In striking contrast to other serpins, miropin efficiently inhibits a broad range of serine (neutrophil elastase, cathepsin G, subtilisin, plasmin, and trypsin) (Ksiazek et al. 2015; Sochaj-Gregorczyk et al. 2020) and cysteine (cathepsin L, Lys-gingipain, Tpr protease) proteases belonging to different clans and families of peptidases and vastly varying in specificity. This broad specificity is achieved through several different reactive sites within the reactive center loop (RCL) instead of one in the typical serpin and unusual plasticity of the protein core to accommodate extra β-strains of variable length (Goulas et al. 2017). Essentially for regulation of proteolytic activity in the periodontal tissue environment, miropin is a lipoprotein located on the T. forsythia surface and fully capable to inhibit Kgp protease on the surface of P. gingivalis the key periodontal pathogen responsible for development of periodontitis and associated systemic diseases. Importantly, for taking advantage of these unusual features we were able to produce the recombinant miropin with remarkable broaden inhibitory spectrum, including apart from neutrophil elastase and Lys-gingipain (Kgp), also Arg-gingipain (Rgp) (FIG. 1).

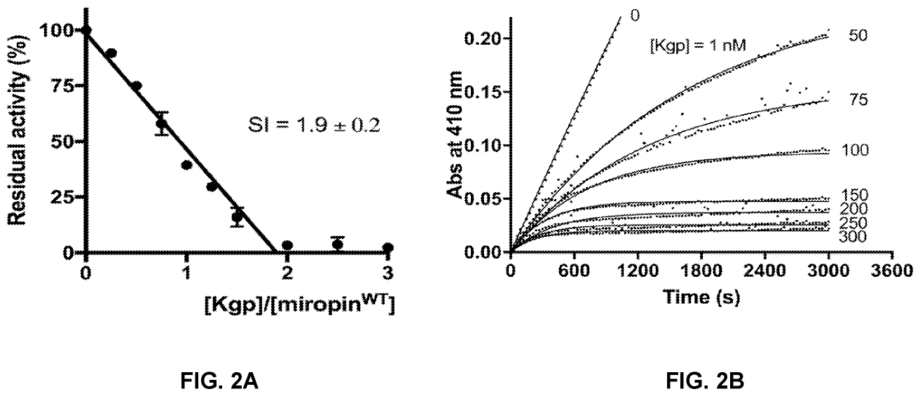

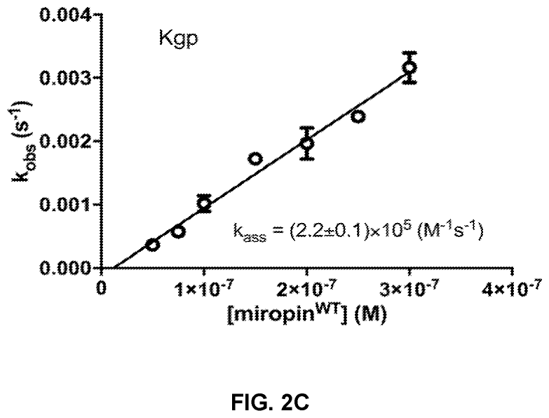

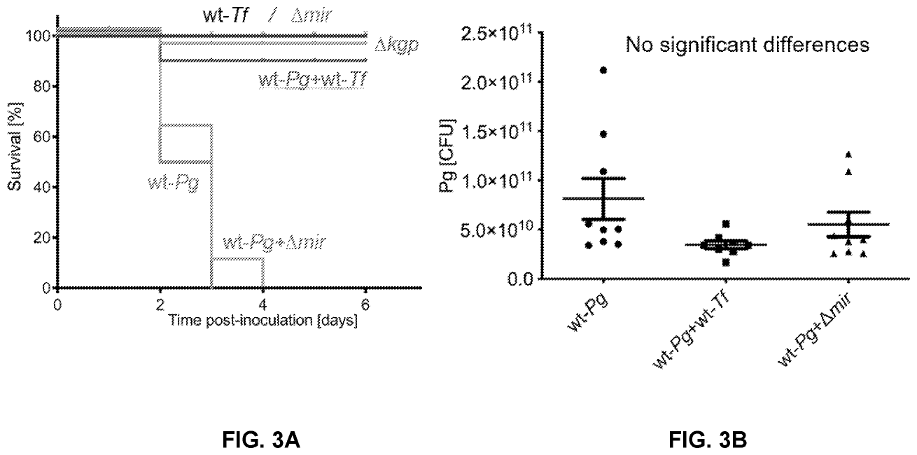

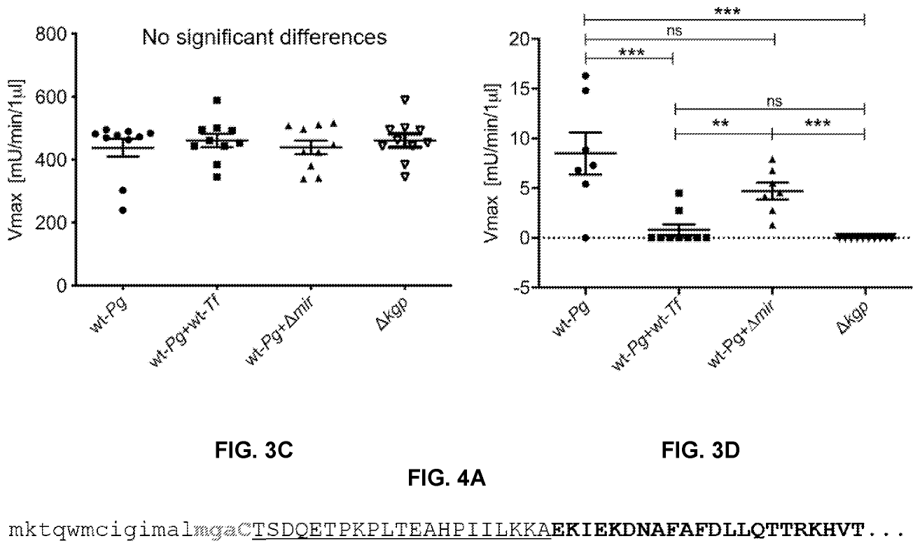

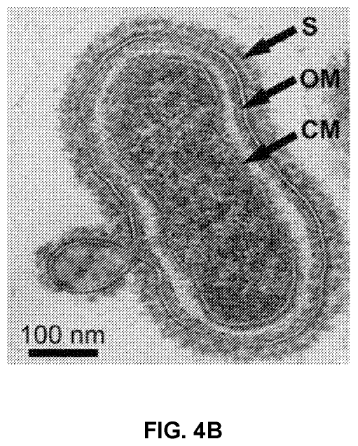

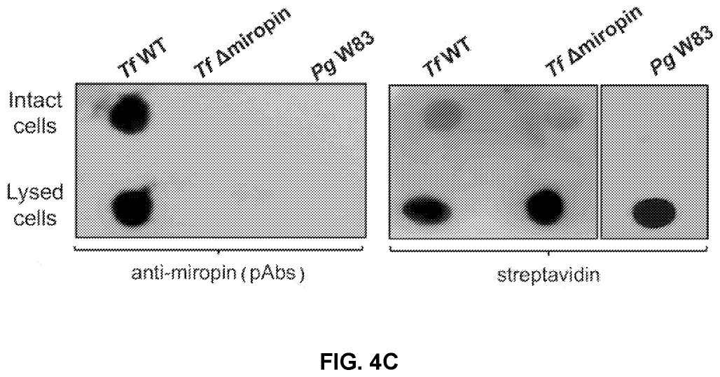

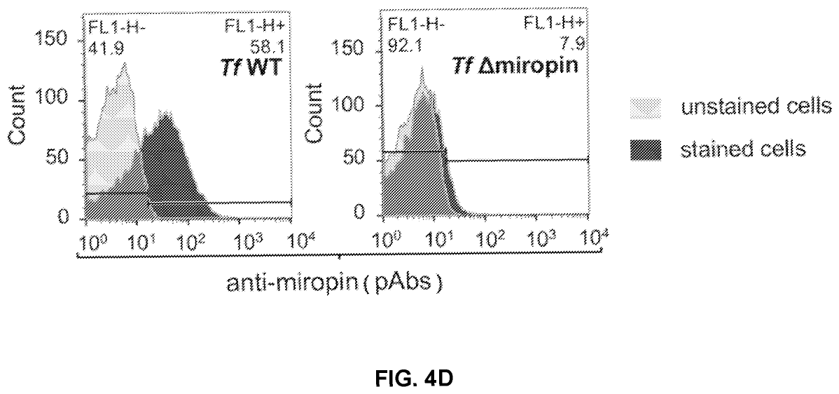

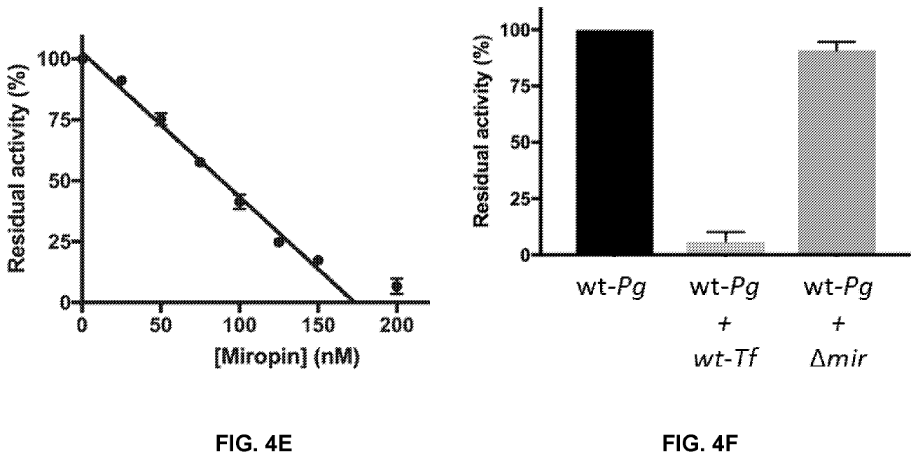

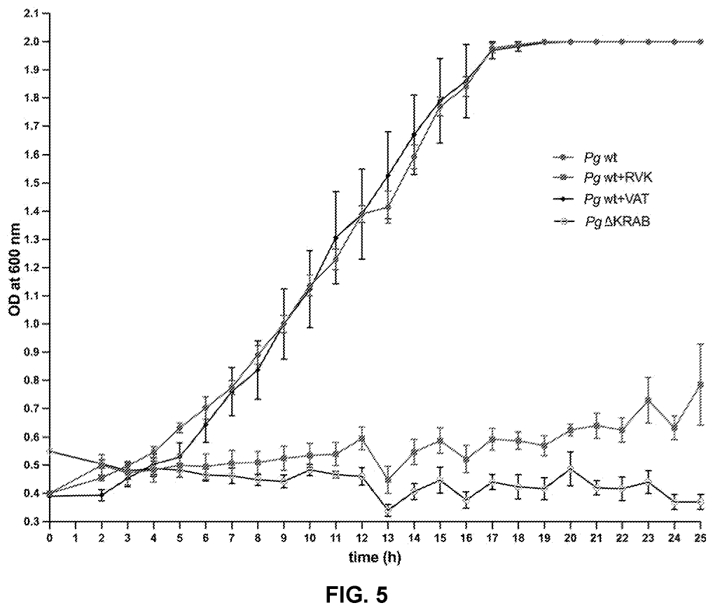

Modified in this way miropin, referred to herein as supermiropin-B) (B for bacteria) blocked P. gingivalis growth in media with albumin as a source of nutritious peptides (FIG. 2) and prevented mortality in mice infected with P. gingivalis (FIG. 3). Furthermore, the miropin gene was mutated in T. forsythia to make the bacterium secreting variants of miropin, including supermiropin-B. Apparently as the wildtype miropin and inhibitor variants are retained in the bacterial surface and can inhibit a variety of human and bacterial proteases (FIG. 4) preventing P. gingivalis growth on minimal medium with albumin as the sole source of nutritious peptides (FIG. 5).

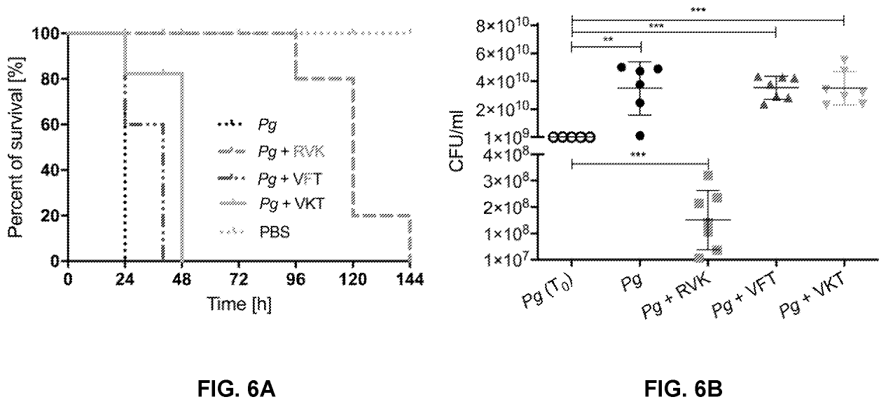

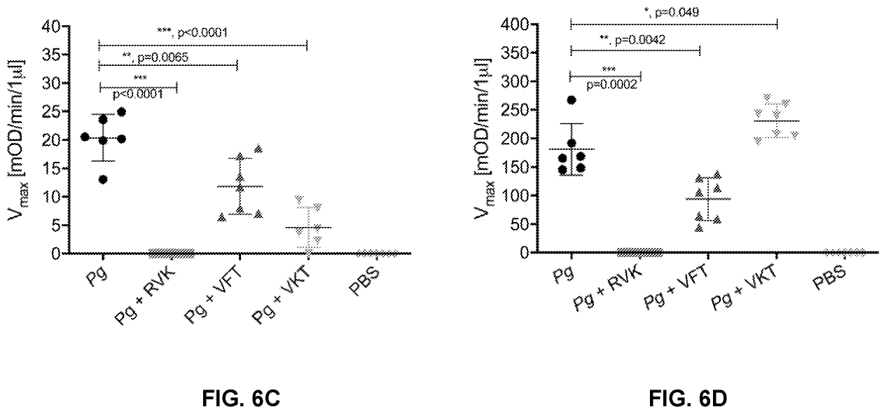

Finally, T. forsythia expressing miropin co-infected with P. gingivalis (Pg) prevented mice mortality caused by the bacterium (FIGS. 6A to 6D). While all mice infected with Pg died within 24 h, injection of miropin variants prevented the mortality to variable degree (FIG. 5A). The least protective effect was exerted by VFT-miropin, which inhibits only Tpr protease of Pg and likely some host proteases but not gingipains. The presence of wt-miropin (VKT), inhibiting Kgp and Tpr protease prolonged survival of 80% of infected mice by 24 h. Finally, RVK-miropin inhibiting both gingipains and Tpr protease exerted long lasting effect. First 2 mice died after 96 h post infection and last 2 animals had to be sacrificed after 144 h. This correlates with the remarkable ability of RVK-miropin to block Pg growth in chambers leading to significant drop of CFU after 24 h. The other variants did not affect Pg proliferation and number of bacteria in chambers increased 40-times during 24 h. The Pg growth arrest by RVK-miropin is clearly associated with total inhibition of both Kgp (FIG. 5C) and Rgp (FIG. 5D) in chambers. Over time both activities slowly increased in parallel with increased density of the Pg population in chambers (data not shown) finally killing mice (FIG. 5A). In contrast to the RVK variant, wt-miropin (VKT) strongly reduced only Kgp activity in keeping with wt-inhibitor specificity. Interestingly, the VFT-variant which has no direct activity against gingipains, slightly but significantly reduced the activity of both gingipains. However, it should be stressed that VFT-variant has almost no effect on survival of Pg infected mice (FIG. 5A).

A Tannerella forsythia strain was obtained expressing modified miropin, RVK, which on the contrary to wild-type (wt) miropin inhibits all major secretory proteases of Porphyromonas gingivalis. The effectiveness of RVK-miropin and the T. forsythia strain expressing this variant of the inhibitor in preventing morbidity and/or mortality of P. gingivalis in different mice models (chamber infection, aspiration pneumonia and oral gavage) was demonstrated.

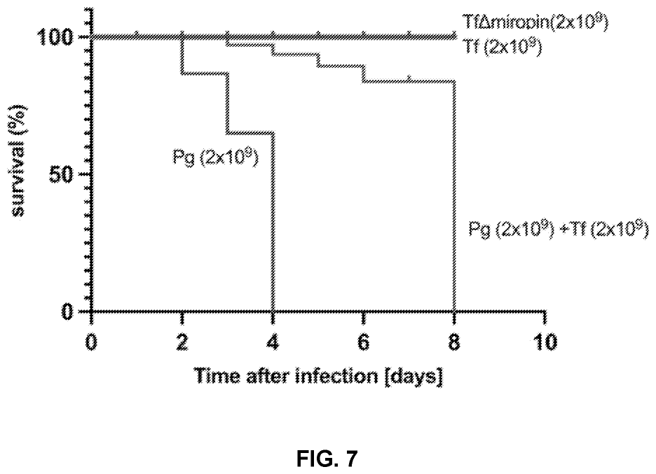

FIG. 7 shows lung co-infection with T. forsythia reduces mice mortality caused by P. gingivalis. Lungs were inoculated with indicated CFU of bacteria and mice survival was recorded.

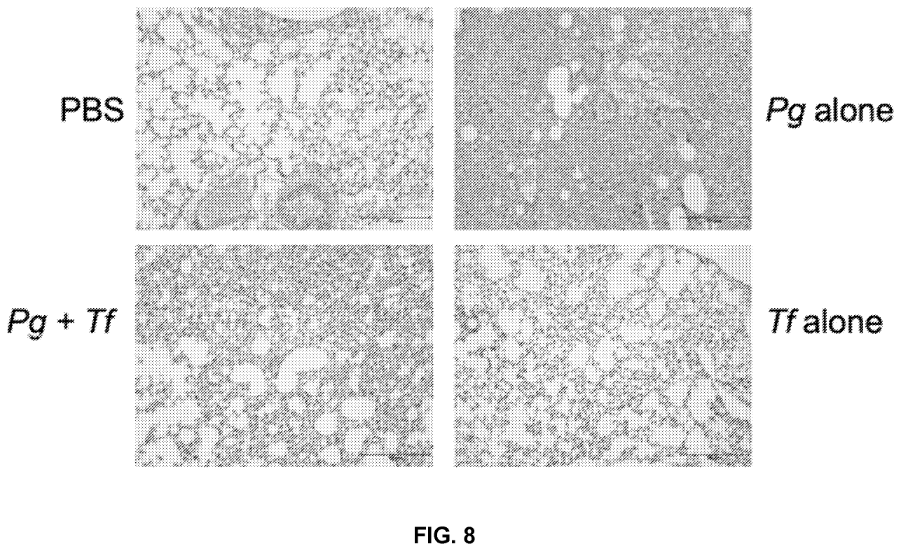

FIG. 8 shows lung co-infection with T. forsythia prevents P. gingivalis-induced lung damage illustrated by histological examination of the lung tissue.

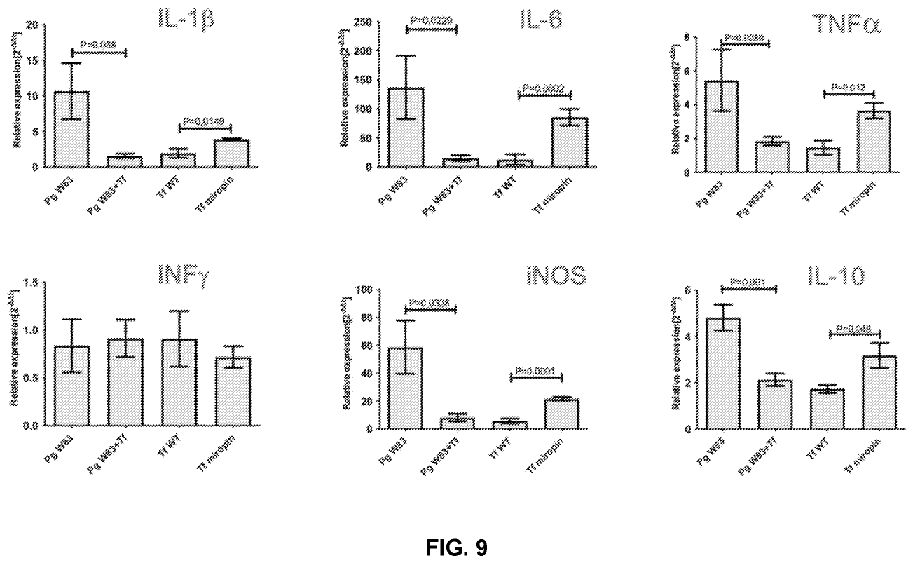

FIG. 9 shows miropin quenches P. gingivalis (Pg)-induced proinflammatory response in infected lungs and contributes to the lack of non-pathogenic character of T. forsythia (Tf). WT=wild type Tf, Tf miropin=, Δmiropin Tf.

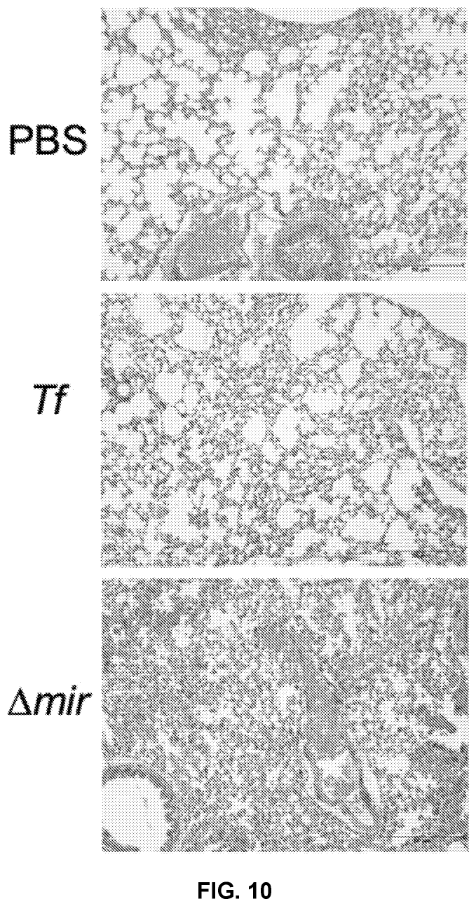

FIG. 10 shows miropin attenuates T. forsythia ability to cause lung damage as illustrated by histological examination of the lung tissue.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A recombinant miropin polypeptide, comprising the amino acid sequence TAVEMX₁X₂X₃SS (SEQ ID NO:11), wherein if X₁ is Val, X₂ is not Lys and/or X₃ is not Thr, and wherein if X₂ is Lys, X₁ is not Val and/or X₃ is not Thr, and wherein if X₃ is Thr, X₁ is not Val and/or X₂ is not Lys, inhibits different proteases than wildtype miropin

2. The recombinant miropin polypeptide of claim 1, comprising the amino acid sequence TAVEMRVKSS (SEQ ID NO:3),

3. The recombinant miropin polypeptide of claim 2, wherein the recombinant miropin comprises the amino acid sequence SEQ ID NO:4, or a variant thereof having at least 65% sequence identity to SEQ ID NO:4.

4. The recombinant miropin polypeptide of claim 1, comprising the amino acid sequence TAVEMVRTSS (SEQ ID NO:5).

5. The recombinant miropin polypeptide of claim 4, wherein the recombinant miropin comprises the amino acid sequence SEQ ID NO:6, or a variant thereof having at least 65% sequence identity to SEQ ID NO:6.

6. The recombinant miropin polypeptide of claim 1, comprising the amino acid sequence TAVEMVARSS (SEQ ID NO:7).

7. The recombinant miropin polypeptide of claim 6, wherein the recombinant miropin comprises the amino acid sequence SEQ ID NO:8, or a variant thereof having at least 65% sequence identity to SEQ ID NO:8.

8. The recombinant miropin polypeptide of claim 1, comprising the amino acid sequence TAVEMVFTSS (SEQ ID NO:9).

9. The recombinant miropin polypeptide of claim 8, wherein the recombinant miropin comprises the amino acid sequence SEQ ID NO:10, or a variant thereof having at least 65% sequence identity to SEQ ID NO:10.

10. The recombinant miropin polypeptide of claim 1, comprising the amino acid sequence TAVEMVETSS (SEQ ID NO:11).

11. The recombinant miropin polypeptide of claim 10, wherein the recombinant miropin comprises the amino acid sequence SEQ ID NO:12, or a variant thereof having at least 65% sequence identity to SEQ ID NO:12

12. The recombinant miropin polypeptide of claim 1, comprising the amino acid sequence TAVEMVDTSS (SEQ ID NO:13).

13. The recombinant miropin polypeptide of claim 8, wherein the recombinant miropin comprises the amino acid sequence SEQ ID NO:14, or a variant thereof having at least 65% sequence identity to SEQ ID NO:14.

14. The recombinant miropin polypeptide of claim 1, comprising the amino acid sequence TAVEMVNTSS (SEQ ID NO:15).

15. The recombinant miropin polypeptide of claim 8, wherein the recombinant miropin comprises the amino acid sequence SEQ ID NO:16, or a variant thereof having at least 65% sequence identity to SEQ ID NO:16.

16. The recombinant miropin polypeptide of claim 1, comprising the amino acid sequence TAVEMVKTSS (SEQ ID NO:18) having at least 1 amino acid substitution.

17. The recombinant miropin polypeptide of claim 16, wherein the recombinant miropin comprises the amino acid sequence SEQ ID NO:17, or a variant thereof having at least 65% sequence identity to SEQ ID NO:17.

18. A method for treating a disease or condition in a subject that involves administering to the subject a recombinant miropin of claim 1.

19. The method of claim 10, wherein the disease or condition is periodontis or pneumonia.

20. (canceled)

21. The method of claim 10, wherein the disease or condition is selected from the group consisting of a2-plasmin-inhibitor deficiency (Miyasato disease), abdominal aortic aneurysm, acute lung injury, acute pancreatitis, acute respiratory distress syndrome, airway, renal, cardiovascular, allergic inflammation, Alzheimer's, aneurism, aortic stenosis, arthritis, asthma, atherosclerosis, autoimmune disease, biocontrol in insect pest control, blood coagulation, bone and joint disorders, cancer, cerebral ischemia, chronic inflammatory lung diseases, chronic obstructive inflammatory diseases, copd, cystic fibrosis, diabetes and obesity, diabetic nephropathy and obesity, diabetic retinopathy, edema, hair loss, hepatic inflammation/fatty liver disease, hereditary angioedema, immune disease, inflammatory airway disease, liver fibrosis, lupus and auto-immune diseases, multiple sclerosis, neurodegenerative disease, neuropathic pain, osteoarthritis, osteoporosis, P. aeruginosa lung infection, pancreatitis, pneumocystis carinii, primary sjogren syndrome, proliferative vitreoretinopathy, psoriasis, rheumatoid arthritis, thrombose, post-transplantation, and viral infection.
